(19) United States
(12) Patent Application Publication
(10) Pub. No.: US 2024/0010964 A1
(43) Pub. Date: Jan. 11, 2024

(54) FLOW GUIDING BARREL FOR IMPROVING GENE TRANSFORMATION EFFICIENCY FOR BIOLISTIC DELIVERY

(71) Applicant: Iowa State University Research Foundation, Inc., Ames, IA (US)

(72) Inventors: Shan Jiang, Ames, IA (US); Kyle Miller, Ames, IA (US); Connor Phelps Thorpe, Ames, IA (US); Alan Eggenberger, Ames, IA (US); Kan Wang, Ames, IA (US)

(21) Appl. No.: 18/348,689

(22) Filed: Jul. 7, 2023

Related U.S. Application Data
(60) Provisional application No. 63/367,932, filed on Jul. 8, 2022.

(51) Int. Cl. C12M 1/42; C12N 15/89

(52) U.S. Cl. CPC C12M 35/00; C12N 15/895

(57) ABSTRACT

Improved biolistic bombardment is observable when using a modification to a double-barrel device. One such modification is a custom attachment that can be retrofit to a standard helium-based gene gun. The custom attachment facilitates DNA and protein delivery. The attachment confines the flow of particles in a barrel similar to the choke on a shotgun barrel.

### CROSS REFERENCE TO RELATED APPLICATIONS

This application claims priority under 35 U.S.C. § 119 to provisional patent application U.S. Ser. No. 63/367,932, filed Jul. 8, 2022. The provisional patent application is herein incorporated by reference in its entirety, including without limitation, the specification, claims, and abstract, as well as any figures, tables, appendices, or drawings thereof.

### STATEMENT REGARDING FEDERALLY SPONSORED RESEARCH OR DEVELOPMENT

This invention was made with government support under Grant Number 2019-67013-29016 awarded by the United States Department of Agriculture (USDA)/National Institute of Food and Agriculture (NIFA). The government has certain rights in the invention.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Jun. 29, 2023, is named “P13909US01_SequenceListing.xml” and is 3,827 bytes in size.

### TECHNICAL FIELD

The present disclosure relates generally to flow guiding barrels for a biolistic particle delivery system.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Genetic engineering in plants is a large field with enormous potential. However, the fundamental tools used by both academia and industry for delivering genetic material to plant genomes are relatively limited, due to the need to circumvent the thick cell wall present in plant cells. While some are able to remove the cell wall to provide easier access, or hijack particular bacteria that have evolved this function, the most general method for doing so is to punch through the barrier using the momentum of a small, dense, DNA- or protein-coated metal particle.

Biolistic particle delivery is a method of transformation that uses helium pressure to introduce DNA-coated microcarriers into cells. Particle delivery transforms intact cells in culture. Microprojectile bombardment can transform such diverse targets as bacterial, fungal, insect, plant, and animal cells and intracellular organelles. Minimal pre- or post-bombardment manipulation is required. Biolistic particle delivery is regarded as being much easier and faster to perform than the tedious task of micro-injection.

This biological-ballistic (biolistic) method has been adopted around the world by university researchers as well as virtually all companies developing genetically modified crops. The main limiting factor when using the biolistic method is low transformation efficiencies. This is due to the small number of cells receiving the DNA/protein upon bombardment and the need to balance the destructive impact with effective cell penetration.

For example, the particle delivery system 100, the PDS-1000/He instrument, which is manufactured and sold by Bio-Rad Laboratories (Hercules, CA; hereinafter “Bio-Rad”), is shown in FIG. 1. The system 100 uses pressurized helium to accelerate subcellular sized microprojectiles coated with DNA or other biological material over a range of velocities necessary to optimally transform many different cell types. A microcarrier launch assembly 200 is placed within a bombardment chamber 102. Access to the bombardment chamber 102 is permitted through a bombardment chamber door 104. The bombardment chamber door 104, with a brace, controls supply of line electrical to the instrument. The bombardment chamber 102 also includes a rupture disk retaining cap 106, a launch assembly shelf 108, and a target plate shelf 300.

The system 100 also includes components for the attachment and delivery of high-pressure helium to the main unit. For example, the system 100 includes vacuum tubing 110 (e.g., reinforced polyvinyl chloride, PVC, tubing) for attachment to a vacuum source. A tank 112 stores pressurized helium. Delivery of helium is controlled by a helium regulator 114, a helium pressure regulator 116, a solenoid valve 118, and connective polyether ether ketone (“PEEK”) tubing 120.

With respect to the controls of the system 100, an on/off power switch 122 controls supply of line electrical power to the instrument. A fire switch 124 controls flow of helium into the gas acceleration tube by activating the solenoid valve 118. The fire switch 124 is illuminated red when enabled. When the safety interlock is satisfied that at least 5″ Hg vacuum is present in the chamber, the fire switch 124 must be held on continuously until rupture disk bursts and then released to stop the flow of helium. If the fire switch 124 is released before the disk ruptures, the helium is vented via a safety vent in the external 3-way metering (solenoid) valve 118. A vacuum/vent/hold switch assembly 126 controls application of vacuum to the bombardment chamber 102. Vacuum is applied from the line source. The vent releases vacuum using filtered air. The hold switch maintains vacuum by isolating chamber. Bombardments are performed with the hold switch in the ‘hold’ position. The vacuum gauge 128 indicates a level of vacuum in the bombardment chamber 102, in inches of mercury where zero equals ambient atmospheric pressure. The vacuum/vent rate control valves 130 regulate rate of application and relief of vacuum in bombardment chamber. Clockwise rotation closes valves. A helium pressure gauge 132 indicates helium pressure (in psi) in the gas acceleration tube.

The microcarrier launch assembly 200 is shown in greater detail in FIGS. 2A-2B. The microcarrier launch assembly 200 is shipped fully assembled, as shown in FIG. 2A. The microcarrier launch assembly 200 comprises the launch assembly shelf 108 with a recessed set screw, a macrocarrier cover lid 202, an adjustable nest 204, a fixed nest 206 with a retaining spring, a stopping screen support ring 208, two spacer rings 210, and five macrocarrier holders 212. The macrocarrier holders 212 are for use within the microcarrier launch assembly 200 after the macrocarrier is inserted using a macrocarrier insertion tool.

The microcarrier launch assembly 200 comprises the launch assembly shelf 300 shown in FIG. 3. The target shelf 300 holds the biological target in a petri plate in the path of the accelerated DNA/microcarrier preparation. Particle flight distance is determined by positioning the shelf at one of four levels using slots in the chamber walls.

More particularly, the particle delivery system 100 shown in FIG. 1 operates using the ballistic process 400 shown in the before and after images shown of FIGS. 4A and 4B. The ballistic process 400 utilizes high-pressure helium 402, released by a rupture disk 404, and partial vacuum (e.g., gas acceleration tube 406) to propel a macrocarrier sheet 408 loaded with millions of microscopic gold microcarriers 410 toward target cells 412 at a high velocity.

The rupture disk retaining cap 106 seals the rupture disk 404 against the chamber end of the gas acceleration tube 406. The rupture disk retaining cap 106 is tightened securely. The microcarrier launch assembly 200 holds the DNA/microcarrier preparation on the macrocarrier sheet 108 over the sheet over the stopping screen in the path of the helium shock wave. When the solenoid valve 118 is activated by the fire switch 124, the needle in the helium pressure gauge 132 (an oil-filled gauge) rotates clockwise until the rupture disk 404 bursts.

The microcarriers 410 are coated with DNA or other biological material for transformation. The macrocarrier 408 is halted after a short distance by a stopping screen 414. The DNA-coated microcarriers 410 continue traveling toward the target cells 412 to penetrate and transform the target cells 412.

The launch velocity of microcarriers 410 for each bombardment depends on the helium pressure (rupture disk 404 selection), the amount of vacuum in the bombardment chamber, the distance 416 from the rupture disk 404 to the macrocarrier sheet 408, the macrocarrier travel distance 418 to the stopping screen 414, and the distance 420 between the stopping screen 414 and the target cells 412.

FIGS. 5A-5B show an example of a portable, helium driven gene gun 500, known as the Helios Gene Gun. The Helios Gene Gun is also manufactured and sold by Bio-Rad. The Helios Gene Gun contrasts the PDS-1000/He instrument because the overall size of the target to be transformed is limited by the size of the chamber and the target tissue is subjected to a vacuum during bombardment. The Helios Gene Gun requires no vacuum and any target accessible to the barrel can be transformed. Consequently, the Helios Gene Gun may be used in a much wider variety of gene transfer applications and provides a tool for both in vitro and in vivo transformations in the research lab. Essentially, any type of cells which can be made accessible to its nozzle may be transformed.

The helium driven gene gun 500 comprises components for preparing DNA-coated microcarriers, coating the DNA-microcarrier suspension onto the inner surface of the tubing, cutting the tubing into cartridges 502 which are used in the helium driven gene gun 500, and finally propel the microcarriers and their associated DNA into cells.

A cylinder lock 504 controls movement of the barrel pin 506. The cylinder lock 504 is spring-loaded. The natural position of the cylinder lock 504 is in the backward (locked) position so that the barrel pin 506 is inserted into the hole in the cartridge holder 508. This keeps the cartridge holder 506 in a proper position for firing. Moving the cylinder lock 504 forward disengages the barrel pin 506 from the cartridge holder 508 to permit removing the cartridge holder from the helium drive gene gun 500. Moving the cylinder lock forward and to the right latches the cylinder lock 506 to permit removal of the cartridge holder 508. However, to prevent damage to the O-rings 510, the cartridge holder 508 is only removed after compressing the cylinder advance lever 512.

The cylinder advance lever 512 is a multi-functional lever that is spring-activated by pulling the cylinder advance lever 512 backwards. When inserting or removing a cartridge holder 508, releasing the cylinder advance lever 512 moves the barrel liner 514 backward, bringing the O-ring 510 on the back of the liner 514 in contact with the cartridge holder 508. After discharging macrocarriers from one cartridge 502, pulling the cylinder advance lever 512 moves the barrel forward to increase the space for inserting the cartridge holder behind the barrel liner 514. This ratchets the cartridge holder 508, bringing the next cartridge 502 into firing position.

A safety interlock switch 516 is held down to permit the trigger button 518 to be operational. Once the interlock switch 516 is depressed, the trigger button 518 is functional for approximately thirty seconds. The LED display 520 flashes quickly during this time. If the trigger button 518 is not pressed within the allotted time, the safety interlock switch 516 must be released and pressed again to re-activate the trigger button 518.

The trigger button 518 controls the flow of helium gas through the gene gun 500. The trigger button 518 acts as a switch and momentarily activates a solenoid to open a main valve, permitting helium to enter the cartridge 502 and barrel. The trigger button 518 only activates for a limited time after the safety interlock switch 516 is depressed.

A metal bar 522 serves as the mechanism that ratchets the cartridge holder 508 from one position to the next when the cylinder advance lever 512 is pressed. When the cylinder advance lever 512 is moved outward prior to inserting a cartridge holder 508, additional room is provided for maneuvering the cartridge holder 508.

The electrical system of the helium driven gene gun 500 is powered by a battery, which is accessed by way of a battery access cover 524. Under normal use, a nine volt (9V) battery should provide sufficient energy for 1000 shots. As shown in FIG. 5B, the battery compartment 526 is in the base of the handle near the attachment fitting 530 for the helium hose. The battery compartment 526 is protected by the battery access cover 524 that slides forward. The battery is inserted with the positive terminal (the smaller of the two terminals 528) facing forward.

FIG. 6A shows how the helium driven gene gun 500 interacts with other major components used for sample delivery, such as the helium regulator 600 (which includes a connection 602 to the helium tank and a sleeve 604) and the helium hose 700. The helium drive gene gun 500 can be prepared for firing, discharging the device, loading cartridges into the cartridge holder 504, and delivering DNA to target cells.

More particularly, the attachment fitting 530 of the helium gun 500 can be inserted into an opening on the body of the female quick-connect fitting 704 on the helium hose 700 and pushed until it clicks or otherwise locks. The helium hose 700 can then be locked into the helium regulator 600 by inserting a stem 702 into the female quick connect fitting 606 on the helium regulator 502 until it clicks or otherwise locks. As a result of the two connections, the helium driven gene gun 500 is indirectly locked into the helium regulator 600. The pressure relief valve 608 can be turned counter-clockwise to depressurize the system in the even the helium regulator 600 has been pressurized prior to the intended time and/or the stem 702 and body 606 will not lock.

In use, and prior to transfection, the plasmid DNA is attached to the gold particles. This is accomplished by precipitation of the DNA from solution in the presence of gold microcarriers and the polycarbon spermidine by the addition of CaCl₂. The particles are then washed extensively with ethanol to remove the water and resuspended in ethanol. The DNA/microcarrier solution is coated onto the inner wall of Gold-Coat tubing and dried. The tubing is cut into 0.5″ length cartridges 502. These cartridges 502, when inserted into the cartridge holder 508 of the helium driven gene gun 500 are the source of the DNA which enters the target cells by the helium discharge.

The helium driven gene gun 500 employs a high velocity stream of helium 610 to accelerate gold particles coated with plasmids or RNA to velocities sufficient to penetrate and transform cells, both in vitro and in vivo, as shown in FIG. 6B. The discharge is initiated by pressing the trigger button 518 which activates the main valve, causing helium to travel down the bore of the particle delivery device (gene gun 500). When the helium enters one of the bores of the cylinder containing the cartridge 502, the gold particles on the inside of the tubing are pulled from the surface, become entrained in the helium stream 610, and begin to pick up speed. Immediately past the acceleration channel 532, the barrel begins to open as a cone. The slope of the cone causes the gas to be pulled outward, expanding the high-pressure stream 610 into a less destructive low velocity pulse, while the gold particles maintain a high velocity. The expansion also helps spread the microcarriers from their original diameter to an area approximately four times greater in diameter at the target site.

Helium gas is pulsed through the cartridge loaded with DNA-coated-microcarriers. This pulse sweeps the microcarriers from the inside wall of the cartridge. As the microcarriers enter the barrel liner 514 they pick up speed in the acceleration channel 532 then spread out as they travel down the barrel; the increased cross-sectional area of the barrel from the acceleration chamber 532 to the spacer 534 also moderates the helium shock wave so it is less intense when it reaches the target cells. The O-rings 510 on each side of the cartridge holder 508 direct the flow of helium 610 through the cartridge 502 and the acceleration channel 532. The spacer 534 maintains target distance and permits venting of the helium gas away from the target.

There exists a need in the art for a device that can dramatically increase the effectiveness of the biolistic particle delivery system and helium driven gene gun described above.

### SUMMARY

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the present disclosure to improve on or overcome the deficiencies in the art.

It is a further object, feature, and/or advantage of the present disclosure to retrofit said device to existing gene gun systems.

It is still yet a further object, feature, and/or advantage of the present disclosure to increase the target area and the rate of cell transfection with a single bombardment, when compared to the target area and the rate of cell transfection of original equipment manufacturers' (OEM) stock components. According to some aspects of the present disclosure, can be accomplished without increasing required DNA, gold, macrocarriers, and/or time. In fact, in some embodiments, the number of required macrocarriers can be reduced from seven to just one.

It is still yet a further object, feature, and/or advantage of the present disclosure to reduce DNA at least seven times (7×), and more preferably at least ten times (10×) with 10% of the delivery agent, while still achieving higher delivery results.

It is still yet a further object, feature, and/or advantage of the present disclosure to use as few materials as possible while still while retaining the efficacy, advantages, and functions of the barrels described herein.

It is still yet a further object, feature, and/or advantage of the present disclosure to intelligently direct air. Flow of the major flow path can be substantially laminar. If the divider(s) are allowed to extend beyond the length of the body, the dividers can help prevent cross-contamination in multiple different samples.

It is still yet a further object, feature, and/or advantage of the present disclosure to be able to 3D print the device. 3D printing can facilitate adapting the device to different plant species and geometries. Alternative forms of additive manufacturing can also be employed, such as injection molding, stereolithography, direct-Energy Deposition, casting, etc.

It is still yet a further object, feature, and/or advantage of the present disclosure to drastically improve the delivery efficiency of existing gene guns and to produce more consistent results. For example, the geometry of the barrel can allow helium flow to confine particles so as to maximize transfection efficiency (cells/area). In some embodiments, use of a gun with the barrel described herein can increase efficiency by a factor of three to four times (7-12×) while reducing a relative standard deviation within the results by approximately fifty percent (50%).

It is still yet a further object, feature, and/or advantage of the present disclosure to enables the application of more DNA solution homogeneously and to more precisely control flow, which can further lead to optimal delivery results.

It is preferred the apparatus be safe, cost effective, and durable. For example, the flow guiding barrel and flow guiding double barrels described herein can be adapted to resist excessive heat, static buildup, corrosion, and/or mechanical failures (e.g. cracking, crumbling, shearing, creeping) due to excessive impacts and/or prolonged exposure to tensile and/or compressive forces acting on the barrels.

At least one embodiment disclosed herein comprises a distinct aesthetic appearance. Ornamental aspects included in such an embodiment can help capture a consumer's attention and/or identify a source of origin of a product being sold. Said ornamental aspects will not impede functionality of the flow-guiding barrel(s).

Methods can be practiced which facilitate use, manufacture, assembly, maintenance, and repair of the flow guiding barrel(s) which accomplish some or all of the previously stated objectives.

The flow-guiding barrels can be incorporated into gene guns at the OEM, other particle moving systems, and/or commercialized separately in kits which accomplish some or all of the previously stated objectives.

According to some aspects of the present disclosure, an attachment for a biolistic particle delivery system that confines the flow of particles to a barrel comprises an elongated body with a central cavity that defines a major flow path passing through; and a ring located at a top of the cylindrical body, said ring having a diameter greater than a diameter of the cylindrical body, wherein the ring is retrofit to a macrocarrier launch assembly of the biolistic particle delivery system. The elongated body can be a hollow cylinder or can taper from an initial thickness greatest at the location where the ring meets the top of the cylindrical body to a terminal thickness less than the initial thickness.

According to some additional aspects of the present disclosure, the major flow path is divided into more than one major flow path by one or more dividers.

According to some additional aspects of the present disclosure, the one or more dividers begin at the top of the cylindrical body and extend only to a depth at or within the elongated body.

According to some additional aspects of the present disclosure, each of the more than one major flow path is defined by a cavity shape having at least one curved wall and at least one planar wall.

According to some additional aspects of the present disclosure, the one or more dividers begin at the top of the cylindrical body and extend to a depth beyond the elongated body, thereby acting as fins to help guide flow even as particles have exited the central cavity.

According to some additional aspects of the present disclosure, the attachment includes minor flow paths located adjacent a periphery of the central cavity, and the major flow path is separated from the minor flow paths by one or more minor dividers (e.g., baffles). Airflow in the device carries particles, which contrasts the OEM stock components that have no or very little airflow that carry such particles. The device therefore allows higher flow rate and better flow capacity.

According to some additional aspects of the present disclosure, the attachment can be symmetrical about at least an x-axis and a y-axis and is asymmetrical about a z-axis (defined by an axial axis traversing a longitude of the elongated body).

According to some other aspects of the present disclosure, a biolistic particle delivery system comprises a gas acceleration tube, a source of high-pressure gas operatively connected to a first end of the gas acceleration tube, and the attachment described by one or more of the preceding paragraphs, said attachment being located at a second end of the gas acceleration tube.

According to some additional aspects of the present disclosure, the biolistic particle delivery system can also include a screen that allows only the biolistic particles to impact an intended target.

According to some additional aspects of the present disclosure, the biolistic particle delivery system comprises a bombardment chamber, connective tubing attached to a vacuum source, a helium regulator, and a solenoid valve. The biolistic particle deliver system can also include a rupture disk.

According to some additional aspects of the present disclosure, the biolistic particle delivery system is a helium driven gene gun.

According to some other aspects of the present disclosure, a method of delivering biolistic particles comprises using a high pressure gas to move biolistic particles through an attachment for a biolistic particle delivery system having an elongated barrel toward a target having one or more cells selected from the group consisting of: bacterial cells, fungal cells, insect cells, plant cells, animal cells, intracellular organelles, and combination(s) thereof; and bombarding the targets with coated microcarriers. The coated microcarriers can deliver DNA, mRNA, viruses, protein, and/or reagents coated on gold particles.

According to some additional aspects of the present disclosure, the method further comprises replacing a stock component with the elongated barrel.

According to some additional aspects of the present disclosure, the method further comprises reducing a number of outlier particles through use of the stock component by employing said elongated barrel in lieu thereof.

According to some additional aspects of the present disclosure, the method further comprises reducing a number of cells killed through use of the stock component by employing said elongated barrel in lieu thereof.

According to some additional aspects of the present disclosure, the method further comprises increasing an efficacy related to penetration of a cell wall and to hit a larger number of cells.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 shows a biolistic particle delivery system.

FIGS. 2A-2B shows a microcarrier launch assembly usable with the biolistic particle delivery system of FIG. 1. FIG. 2A shows an assembled view. FIG. 2B shows a disassembled view.

FIG. 3 shows an elevation view of a target shelf usable with the biolistic particle delivery system of FIG. 1.

FIG. 4 shows the biolistic bombardment process using the biolistic particle delivery system of FIG. 1.

FIG. 5A shows a side elevation view components and controls on a helium driven gene gun.

FIG. 5B shows a side, partial cutaway view of a battery compartment of the helium driven gene gun of FIG. 5A.

FIG. 6A shows an exploded view of major components used for sample delivery with the helium driven gene gun of FIG. 5A.

FIG. 6B shows a cross-sectional view of a helium driven gene gun, emphasizing view of a high velocity stream of helium that accelerates gold particles coated with plasmids or RNA to velocities sufficient to penetrate and transform cells, both in vitro and in vivo.

FIG. 7 shows a schematic view of a helium driven gene gun, including comparison views of a stock component within the helium driven gene gun versus use of the flow guiding barrel(s) described herein.

FIG. 8A shows a front elevation view of single-flow guiding barrel, according to some aspects of the present disclosure. FIG. 8B shows a rear elevation view thereof. FIG. 8C shows a bottom perspective view thereof. FIG. 8D shows a top perspective view thereof. FIG. 8E shows a side cross section view thereof. FIG. 8F shows a side elevation view thereof. FIG. 8G shows a top cross section view thereof. FIG. 8H shows a top plan view thereof. FIG. 8I shows a top perspective view thereof. FIG. 8J shows a front perspective view thereof. FIG. 8K shows a bottom plan view thereof.

FIG. 9A shows a front elevation view of double-flow guiding barrel, according to some aspects of the present disclosure. FIG. 9B shows a rear elevation view thereof. FIG. 9C shows a top perspective view thereof. FIG. 9D shows a front perspective view thereof. FIG. 9E shows a side elevation view thereof. FIG. 9F shows a top cross section view thereof. FIG. 9G shows a top plan view thereof. FIG. 9H shows a side cross section view thereof. FIG. 9I shows a bottom plan view thereof. FIG. 9J shows a bottom perspective view thereof. FIG. 9K shows a side perspective view thereof.

FIG. 10A shows a perspective view of multi-flow guiding barrel, according to some aspects of the present disclosure. FIG. 10B shows a front elevation view thereof. FIG. 10C shows a rear elevation view thereof. FIG. 10D shows a left-side elevation view thereof. FIG. 10E shows a right-side elevation view thereof. FIG. 10F shows a top plan view thereof. FIG. 10G shows a bottom plan view thereof.

FIG. 11A shows variances in barrel length and diameter for the flow-guiding barrel(s), according to some aspects of the disclosure.

FIG. 11B illustrates the effect of in barrel length for the stock component included in the OEM gene gun of FIG. 1 as compared to the improved gene gun of FIGS. 7-8 that incorporates a flow-guiding barrel(s).

FIG. 12 illustrates the effect of variances in barrel diameter for the stock component included in the OEM gene gun of FIG. 1 as compared to the improved gene gun of FIG. 7 that incorporates a flow-guiding barrel(s).

FIG. 13A charts a demonstration of improvement using the flow guiding barrel and quantifies adjacent thereto a total number of transformed cells for both the flow guiding barrel and the stock component. Onion epidermis cells were bombarded with 24 ng of a plasmid that expresses green fluorescent protein and were imaged after two days. Cells were counted via CellProfiler software, as shown in FIG. 13B (for the stock component) and FIG. 13C (for the flow guiding barrel).

FIG. 14A charts the number of cells transformed using either the stock component (n=23) with 100% of the DNA and spermidine and the novel barrel extension (n=18) with 10% of the DNA and spermidine. Onion epidermis cells were bombarded with 2.4 ng and 24 ng of a plasmid that expresses green fluorescenct protein and were imaged after two days. Cells were counted via CellProfiler software, as shown in FIG. 14B (for the stock component) and FIG. 14C (for the flow guiding barrel).

FIG. 15A charts transient protein delivery improvements using a modified barrel. Onion epidermis cells were bombarded with 30 ug of fluorescein isothiocyanate (FITC) labelled bovine serum albumin (BSA) and were imaged after two days. Cells positive with FITC marker were counted via CellProfiler software, as shown in FIG. 15B (for the stock component) and FIG. 15C (for the flow guiding barrel).

FIGS. 16A-16C show a schematic representation of two plasmids used for the evaluation of CRISPR reagents. FIG. 16A shows CRISPR plasmid pTF6005 that carries a Cas9 expression cassette under the control of maize ubiquitin promoter and Cauliflower Mosaic Virus (CaMV) 35S terminator (T35S); OsPDS gRNA1 is regulated by OsU6 promoter; hygromycin resistance gene (hpt II) is driven by 2×CaMV 35S promoter (P35S) and terminated by T35S. RB, T-DNA right border; LB, T-DNA left border; SpR, spectinomycin resistance gene; ColE1 ori, high copy number origin of replication for E. coli; pVS1, origin of replication from plasmid VS1 for Agrobacterium. FIG. 16B shows the reporter plasmid pKL2187 has genes for the red fluorescent protein tdTomato and the green fluorescent protein ZsGreen1. Transcription of the tdTomato gene is driven by a 2× P35S and terminated by an Agrobacterium nopaline synthase terminator (Tnos). The encoded tdTomato protein has an SV40 nuclear localization signal at the N terminus. Transcription of the ZsGreen1 gene is driven by a 2× P35S promoter and terminated by a potato protease inhibitor II terminator (TpinII). The translation start codon is preceded by a TMV Q translational enhancer and is immediately followed by the target sequence of the OsPDS-gRNA1 (SEQ ID NOs: 1 and 2) expressed from pTF6005. The open reading frame for the flexible peptide linker 2× (GGGGS) and ZsGreen1 is out-of-frame by 1-bp with the start codon and is not translated; however, indel mutations at the gRNA target site can bring the ZsGreen1 gene in-frame and restore green fluorescence. AmpR, ampicillin resistance gene. The plasmid pKL2188 is identical to pKL2187 except that the ZsGreen1 gene is in-frame with the start codon. Blue letters, gRNA target sequence; underscored red letter, PAM sequence. FIG. 16C shows the OsPDS target site (SEQ ID NO: 1) as well as one on-target gRNA tested for editing efficiency. The gRNA1 (SEQ ID NO: 3) was driven by OsU6 promoter in the CRISPR plasmid shown in FIG. 16A.

FIG. 16D charts improved cas9-plasmid delivery and editing efficiency using a modified barrel. Onion epidermis cells were bombarded with 2 ug a CRISPR-Cas9 plasmid construct with OsPDS gRNA1, called pTF6005 as well as a reporter plasmid pKL2197. The onions were incubated at 30° C. in dark for 2 days prior to imaging. These constructs carry a gRNA targeting the OsPDS gRNA1 target site in pKL2187 and are expected to result in GFP expressing cells if cas9 editing occurred. Cells were counted via CellProfiler software, as shown in FIG. 16E (for the stock component) and FIG. 16F (for the flow guiding barrel).

FIG. 17A charts improved viral-plasmid delivery using Soybean Mosaic Virus expressing green fluorescent protein in Soybean using a modified barrel. Two-week old juvenile soybean plants were bombarded with 2 ug of SMV plasmid expressing green fluorescent protein and then kept in the green house for a remaining two weeks. After two weeks, the soybean inoculated leaves as well as newly grown leaves were imaged for gfp-expression and reverse transcription-polymerase chain reaction (rt-pcr) to confirm the viral infection, as shown in FIG. 17B (stock component) and FIG. 17C (flow guiding barrel).

FIG. 18A charts improved viral-plasmid delivery using Sugarcane Mosaic Virus expressing green fluorescent protein in Corn using a modified barrel. Two-week old juvenile corn plants were bombarded with 2 ug of SCMV plasmid expressing green fluorescent protein and then kept in the green house for a remaining two weeks. After two weeks, the corn inoculated leaves as well as newly grown leaves were imaged for gfp-expression and reverse transcription-polymerase chain reaction (rt-pcr) to confirm the viral infection, as shown in FIG. 18B (stock component) and FIG. 18C (flow guiding barrel).

FIG. 19A charts improved penetration depth (i.e., velocity) using a modified barrel. FIG. 19B charts target area/particle distribution using a modified barrel. 1 wt % Agarose gels were bombarded with 30 ug of fluorescein isothiocyanate (FITC) labelled bovine serum albumin (BSA) and were imaged under a fluorescent confocal microscope immediately after to determine particle penetration and distribution. Particles were counted via CellProfiler software, as shown in FIG. 19C (for the stock component) and FIG. 19D (for the flow guiding barrel).

FIG. 20 aggregates views of various barrels. The first column of FIG. 20 shows various single barrels, including a perspective view of a stock component, a perspective view of a short, flow-guiding single barrel, and a perspective view of a long, flow-guiding single barrel. The second column of FIG. 20 shows various double barrels, including a perspective view of a narrow double barrel, a perspective view of wide double barrel, and a perspective view of a double barrel with a single fin. The third column of FIG. 20 shows various multi-barrels, including a perspective view of a multi-barrel, a perspective view of a short, flow-guiding multi-barrel, and a perspective view of a long, flow-guiding multi-barrel. The fourth column of FIG. 20 shows various multi-flow barrels, including a perspective view of a flow-guiding multi-flow barrel, a perspective view of a flow-guiding multi-flow barrel having a single fin, and a perspective view of a flow-guiding multi-flow barrel having a plurality of fins.

FIG. 21A compares use of a known barrel versus use of a flow-guiding double-barrel with respect to a total number of transformed cells and performance ratios. FIG. 21B charts the results of testing a double-barrel to the flow guiding double-barrel described herein. The first chart shows an improvement in number of cells transfected using the new design. The second chart measures the ability of the double-barrel to evaluate a consistent performance ratio, i.e. the number of cells transfected via the right barrel compared to the left. A performance ratio of 1 presents the real results, as both sides should be identical. The 3D images show the different barrel designs.

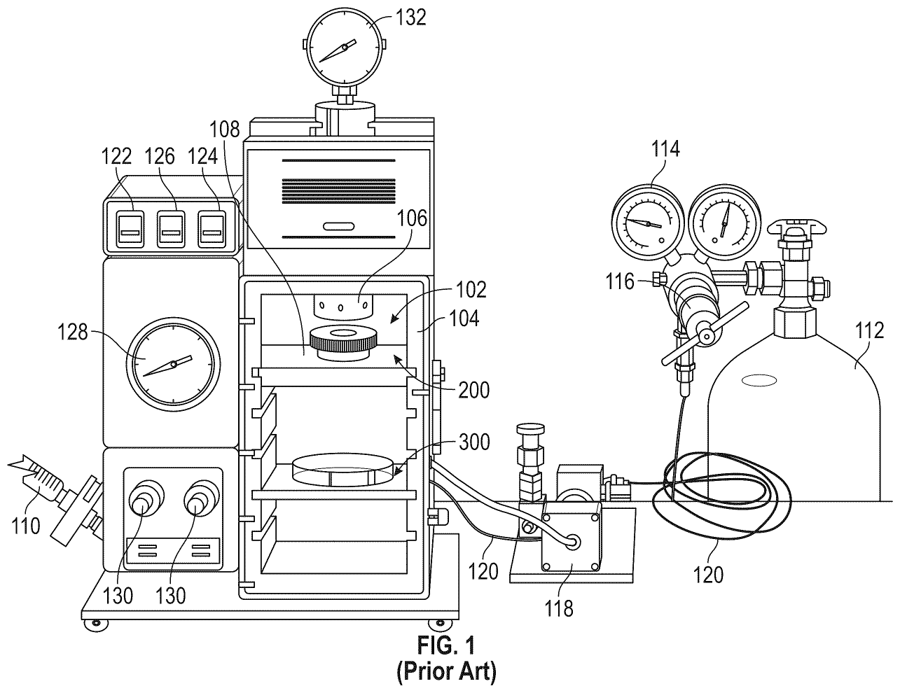

An artisan of ordinary skill in the art need not view, within isolated figure(s), the near infinite number of distinct permutations of features described in the following detailed description to facilitate an understanding of the present disclosure.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

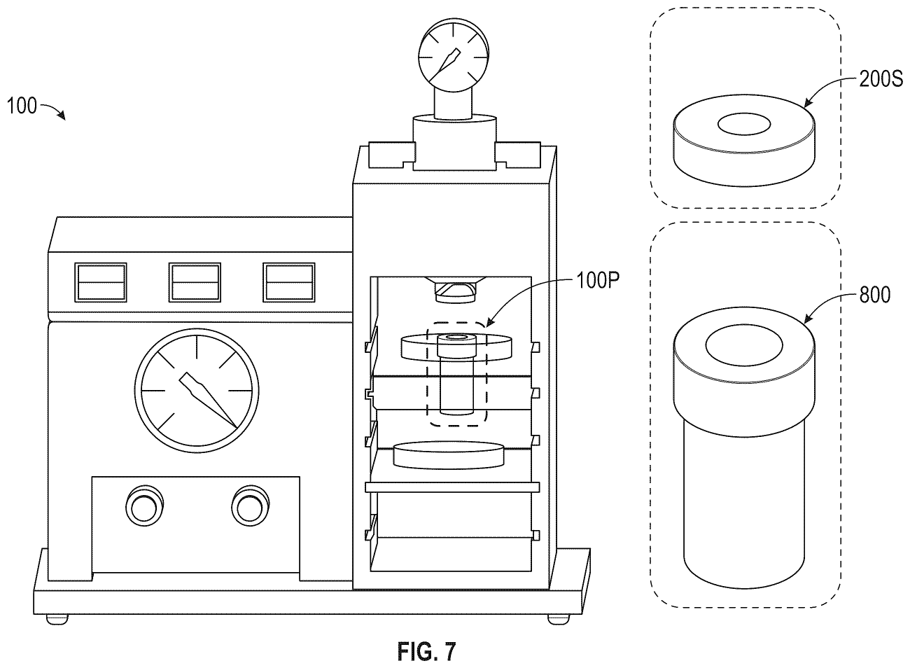

FIG. 7 shows a diagram illustrating the improvements that can be implemented with the helium-based gene gun system 100. The stock component 200S includes the macrocarrier cover lid 202, adjustable nest 204, fixed nest 206 with a retaining spring, stopping screen support ring 208, two spacer rings 210, and five macrocarrier holders 212 described above. The stock component 200S can be swapped out in favor of any one or more of the improved flow guiding barrels described herein 800, 900, 1000. This exchange of components can occur at position 100P within the bombardment chamber 102.

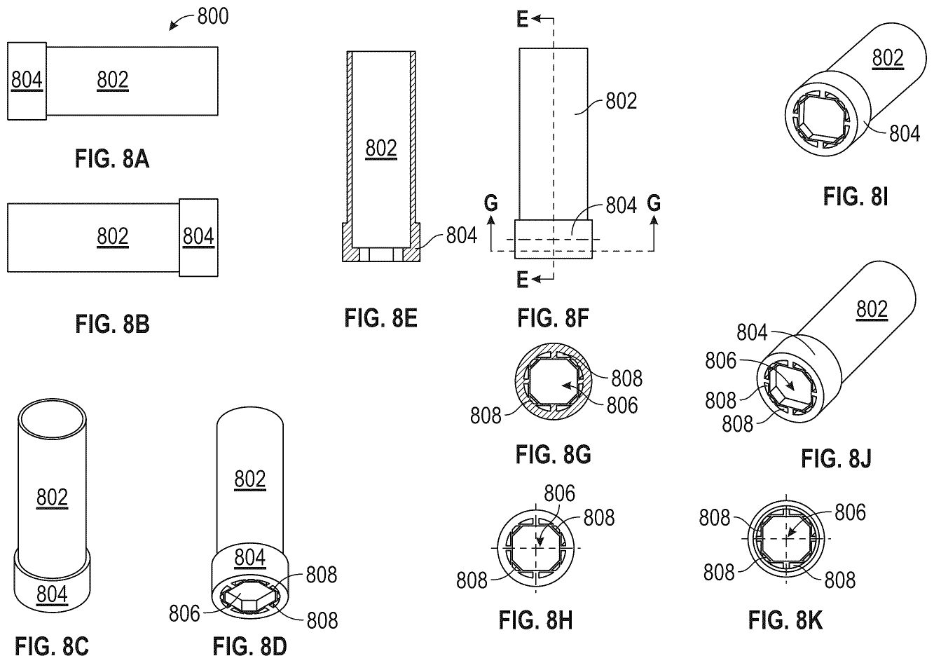

FIGS. 8A-8K depict a single-flow guiding barrel 800. The barrel 800 comprises a body 802, a retaining ring 804, a single major flow path 806, and optionally, several minor, peripheral flow paths 808 defined by baffles located near the periphery of a central cavity.

Holes on the barrel 800 wall help release the pressures and bombard taller plants. The materials of the barrel 800 are not limited to hard plastics. For example, the barrel 800 can be made from an inert material constructed to withstand the pressure used within the system.

The retaining ring 804 acts as a head and includes a larger diameter than that of the barrel 800 so that the barrel 800 does not slide through the holder. The length of the barrel 800 can vary, however in some embodiments is at least four times (4×) greater than the thickness of the retaining ring.

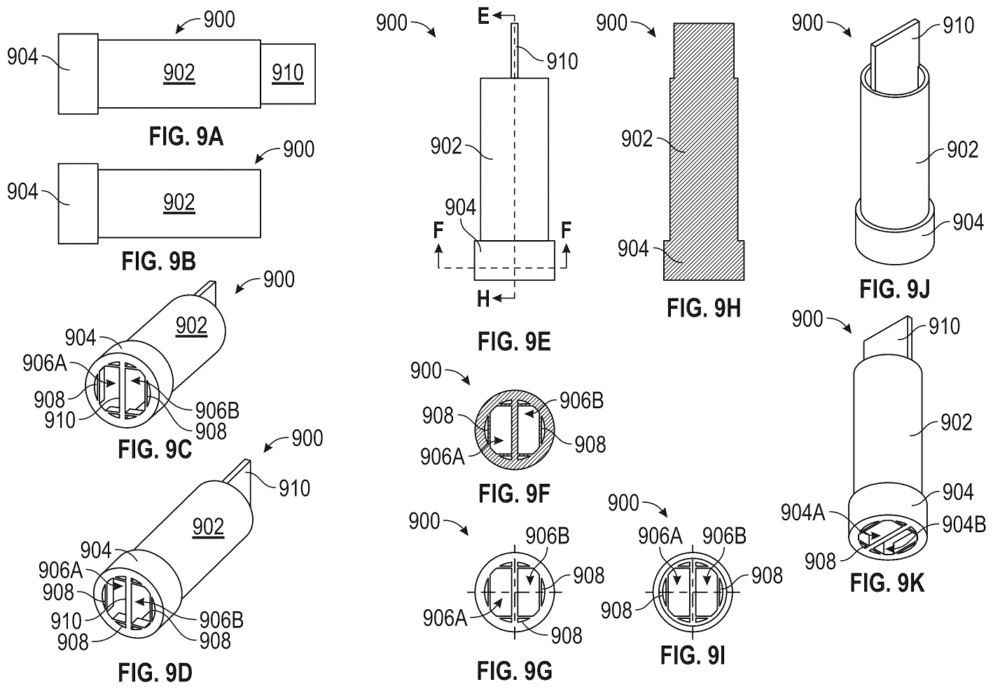

FIGS. 9A-9K depict a double-flow guiding barrel 900. The barrel 900 comprises a body 902, a retaining ring 904, major flow paths 906A & 906B, optionally: several minor, peripheral flow paths 908 defined by minor dividers (e.g., baffles) located near the periphery of the central cavities, and a major divider 910 that separates the major flow paths 906A & 906B. As shown, the major divider can also extend beyond the body 902 and act as a flow guiding fin to deliver particles to a location more proximate the plant tissue.

Holes on the barrel 900 wall help release the pressures and bombard taller plants. The materials of the barrel 900 are not limited to hard plastics. For example, the barrel 900 can be made from an inert material constructed to withstand the pressure used within the system.

The retaining ring 904 acts as a head and includes a larger diameter than that of the barrel 900 so that the barrel 900 does not slide through the holder. The length of the barrel 900 can vary, however in some embodiments is at least four times (4×) greater than the thickness of the retaining ring.

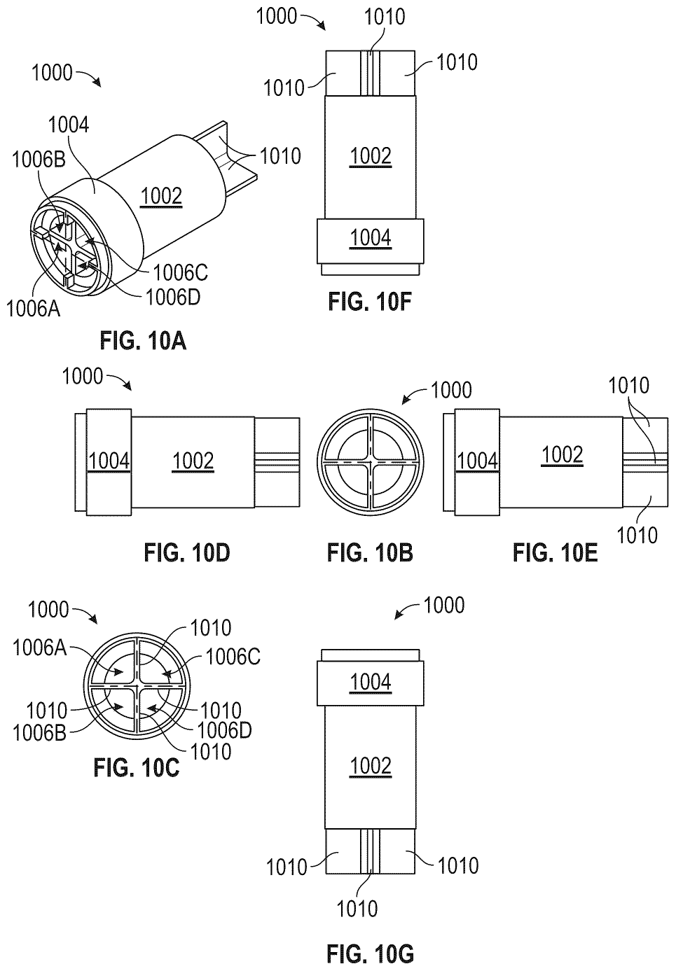

FIGS. 10A-10G depict a multi-flow guiding barrel 1000. The barrel 1000 comprises a body 1002, a retaining ring 1004, major flow paths 1006A-1006D, optionally: several minor, peripheral flow paths (not shown) defined by minor dividers (e.g., baffles) located near the periphery of the central cavities, and major dividers 1010 that separates the major flow paths 1006A-1006D. As shown, the major dividers 1010 can also extend beyond the body 1002 and act as a flow guiding fins to deliver particles to a location more proximate the plant tissue.

Holes on the barrel 1000 wall help release the pressures and bombard taller plants. The materials of the barrel 1000 are not limited to hard plastics. For example, the barrel 1000 can be made from an inert material constructed to withstand the pressure used within the system.

The retaining ring 1004 acts as a head and includes a larger diameter than that of the barrel 800 so that the barrel 1000 does not slide through the holder. The length of the barrel 1000 can vary, however in some embodiments is at least four times (4×) greater than the thickness of the retaining ring.

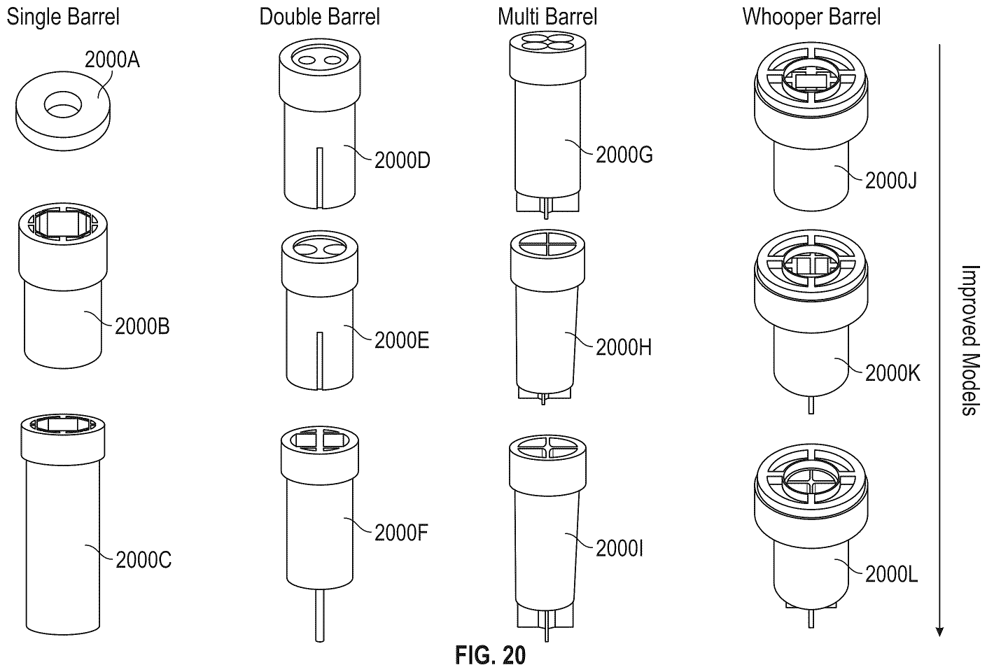

Reverse configurations can also exist (see e.g., barrels 2000D-E of FIG. 20), wherein the body of the barrels 800, 900, 1000 can extend well past where the dividers 910, 1010 terminate and therefore leave what looks to be a cutout in its stead.

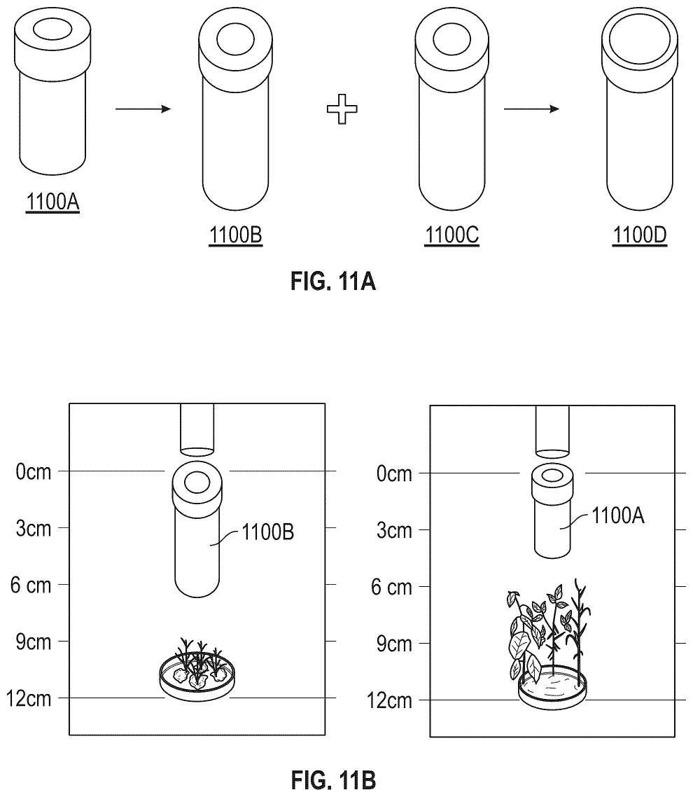

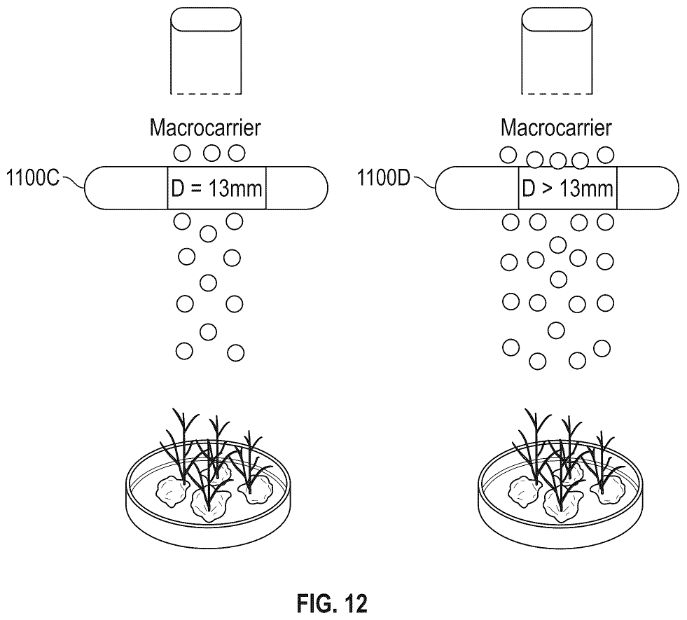

Particles are accelerated through a burst of pressurized gas from the top of the gun, through the barrel 800/900/1000, toward plant tissue. As shown in FIGS. 11A-11B, FIG. 12, and barrels 2000A-L of FIG. 20, the length and inner diameter of the barrel 800 have a great effect on the efficacy of the gene gun 800.

FIG. 20 further depicts a stock component 2000A that comes with the gene gun 800 in the upper left-hand corner. The stock component 2000A is essentially a metal ring that particles travel through after being accelerated by a burst of pressurized gas, and its function is to support a screen that allows only particles to impact the plant tissue. The metal ring is a rudimentary form of a single barrel but does not have the length sufficient to guide flow in order to best transfect cells.

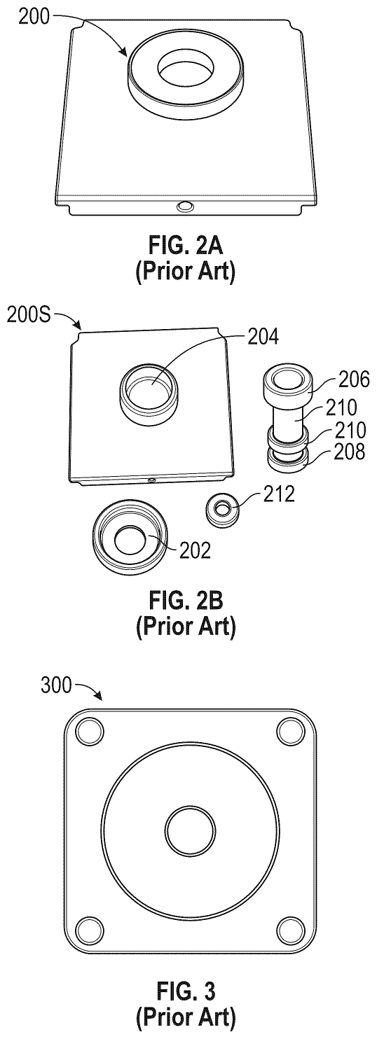

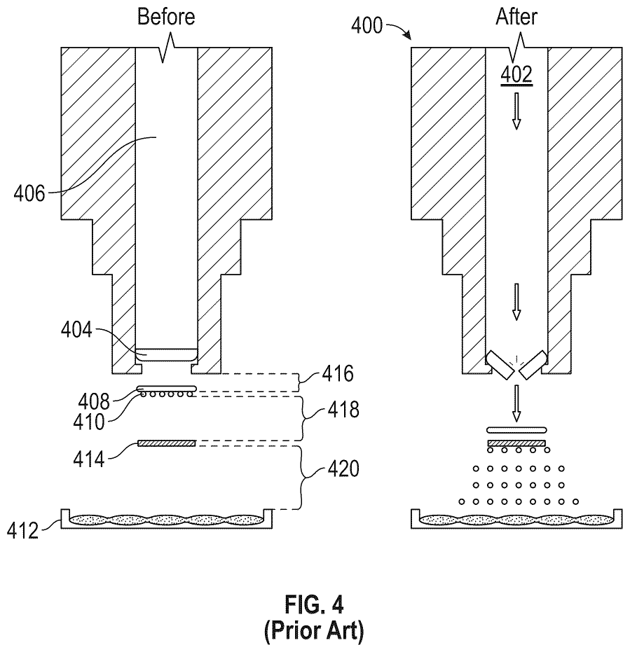

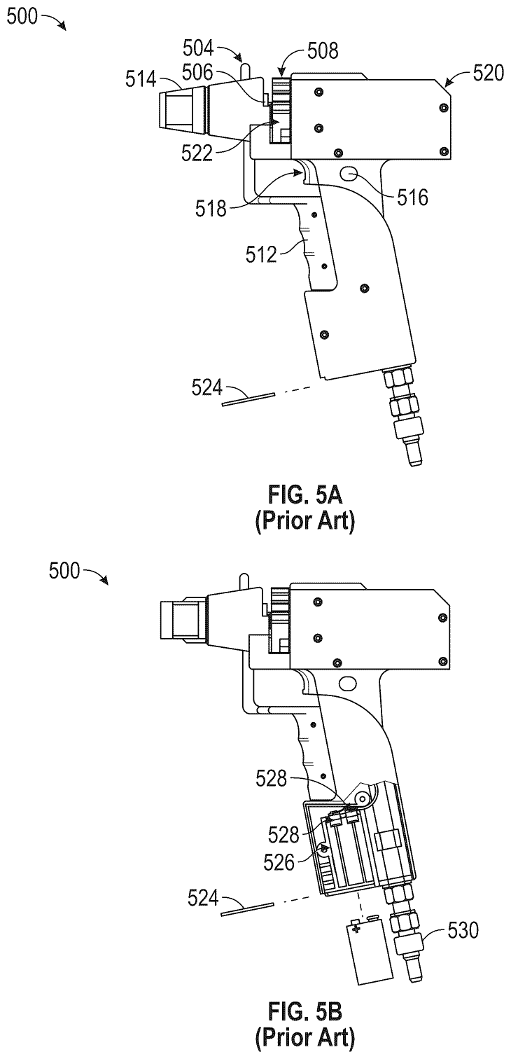

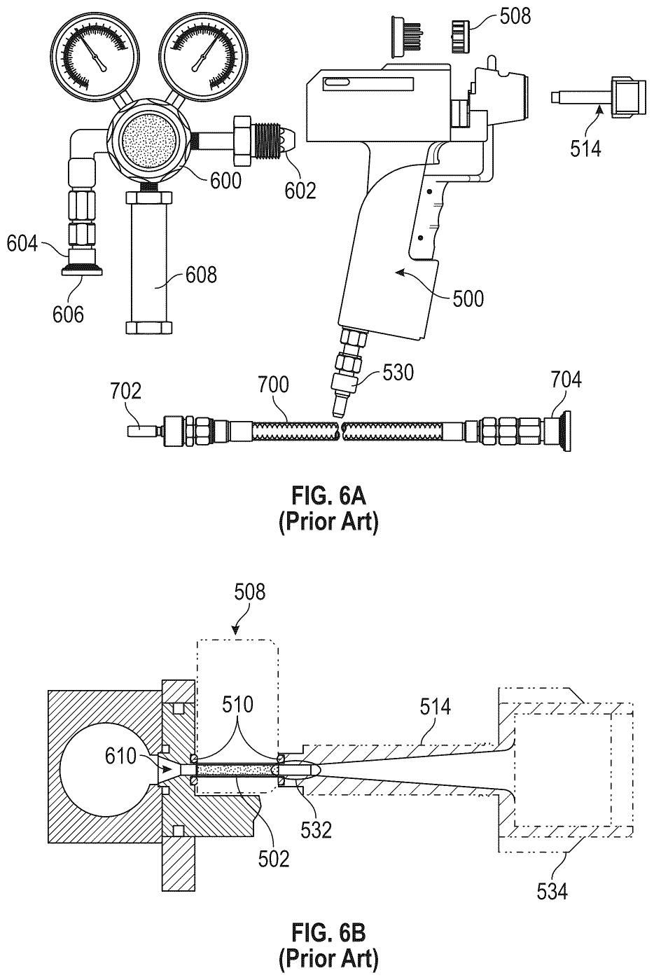

The remaining barrels 2000B-L show barrels (including some that were shown in FIGS. 8A-8K, 9A-9K, and 10A-10G) that deviate from the OEM design shown in FIG. 2. The barrels retrofit to the biolistic particle delivery system 100 and can replace the stock component entirely.

In operation, the gene gun 100 accelerates particles via a burst of pressurized gas (e.g., helium, nitrogen, etc.) from the top of the gun. The particles are sent through the barrel 800 and toward plant tissue. The length and inner diameter of the barrel 800 can be critical factors in its improved effectiveness over the stock component.

The barrels 800, 900, 1000 themselves are an attachment for a helium-based gene gun 100 that confines the flow of particles to a barrel similar to rifling on a traditional firearm. The stock component 200S is a metal ring that particles travel through after being accelerated by a burst of pressurized gas, and its function is to support a screen that allows only particles to impact the plant tissue. The flow guiding barrel attachment has been designed to replace this ring and not only performs its function, but more uniformly directs the airflow to the plant tissue. As the DNA-loaded particles and burst of pressurized gas flow through the device, the confinement of the flow guiding barrel reduces turbulence in the chamber. The major flow path within the cavity of the barrel is therefore substantially laminar. This leads to fewer outlier particles that either kill the cells with their impact or lose too much momentum and are unable to penetrate the cell wall.

From the foregoing, it can be seen that the present disclosure accomplishes at least all of the stated objectives.

### EXAMPLES

The inventors tested the barrel 800 with one of the most common gene guns used in industry today, the Bio-Rad PDS-1000/He™ Biolistic Particle Delivery System. The barrel 800 replaced the stock component 200. The flow guiding barrel was produced via 3D printing and the dimensions and material can be customized to other gene gun systems.

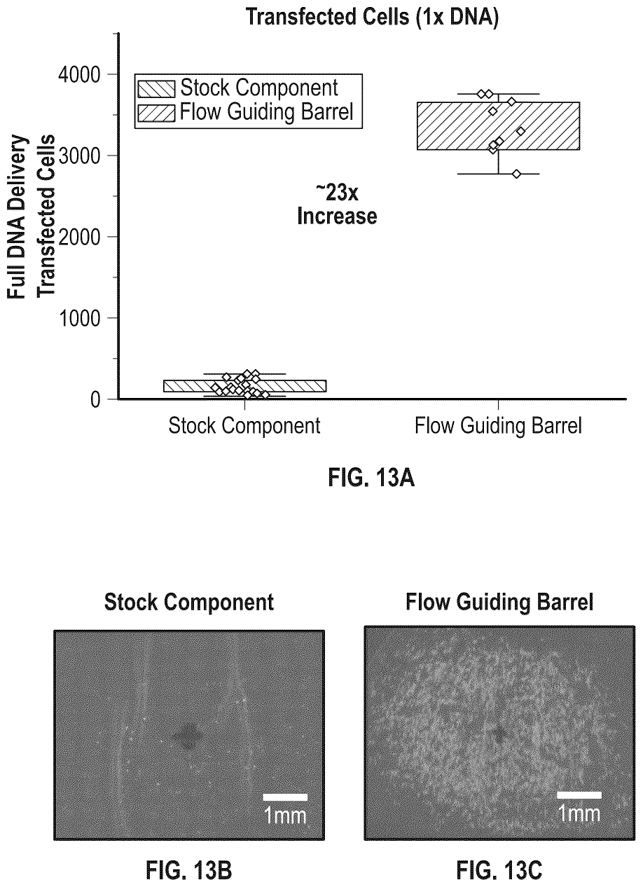

As shown in FIGS. 13A-13C, on average, using the flow guiding barrel leads to at least 23 times (23×) more cells modified per shot (153 cells compared to 3324 cells on average). This was tested using onion epidermis cells bombarded with plasmid DNA that expresses a green fluorescent protein (GFP). The critical parameters of DNA and particle amount were kept constant for each bombardment, and plant tissue was sourced from the same onions. The distance between onion tissue and initial particle position was optimized for both conditions to account for the acceleration generated by increased confinement. The improvement in performance due to the flow guiding barrel represents a significant upgrade that has broadly applicable potential.

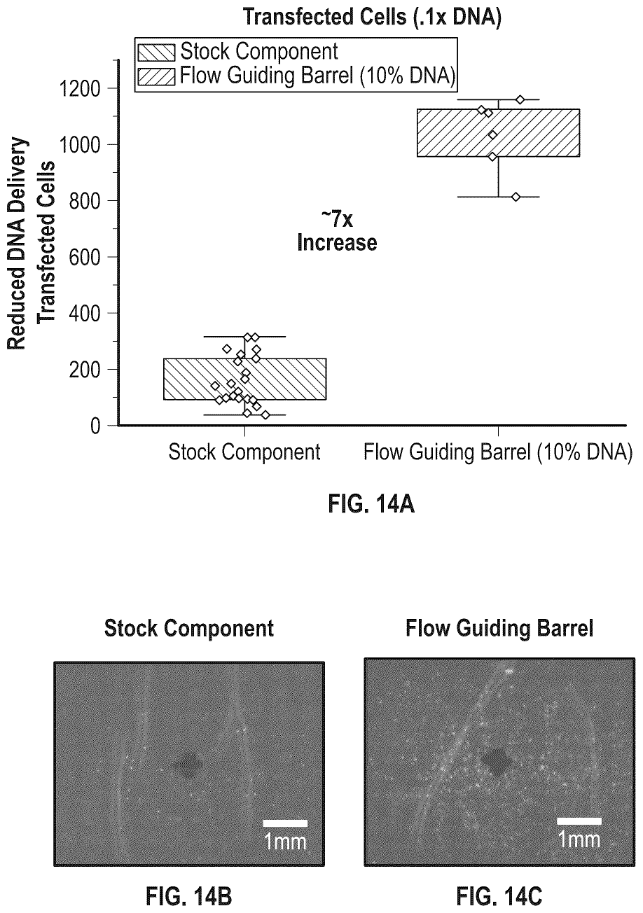

As shown in FIGS. 14A-C, on average, using the flow guiding barrel with 10% of the DNA and delivery agent leads to at least 7 times (7×) more cells modified per shot (153 cells compared to 1030 cells on average). This was tested using onion epidermis cells bombarded with plasmid DNA that expresses a green fluorescent protein (GFP). The critical parameters of DNA and particle amount were kept constant for each bombardment, and plant tissue was sourced from the same onions. The distance between onion tissue and initial particle position was optimized for both conditions to account for the acceleration generated by increased confinement. The improvement in performance using minimal amounts of DNA due to the flow guiding barrel represents a significant upgrade that can potentially help reduce the number of DNA copies inserted.

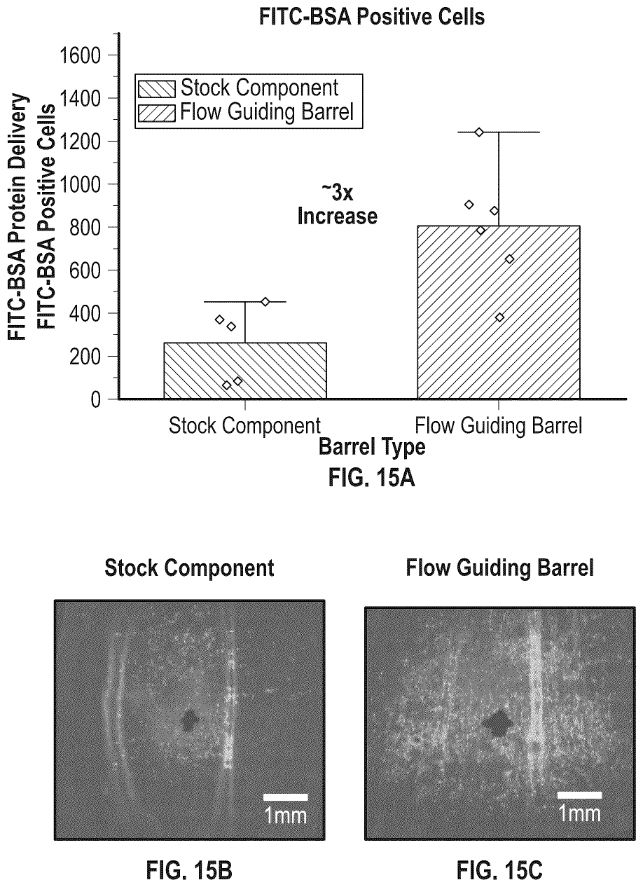

As shown in FIGS. 15A-C, on average, using the flow guiding barrel leads to at least 3 times (3×) more cells distributed with protein (259 cells compared to 807 cells on average). This was tested using onion epidermis cells bombarded with FITC labeled BSA to track delivery. The critical parameters of protein and particle amount were kept constant for each bombardment, and plant tissue was sourced from the same onions. The distance between onion tissue and initial particle position was optimized for both conditions to account for the acceleration generated by increased confinement. The improvement in performance and distribution using protein due to the flow guiding barrel represents a significant upgrade that can broadly help improve protein delivery including Cas9, a CRISPR-associated (“Cas”) endonuclease, or enzyme.

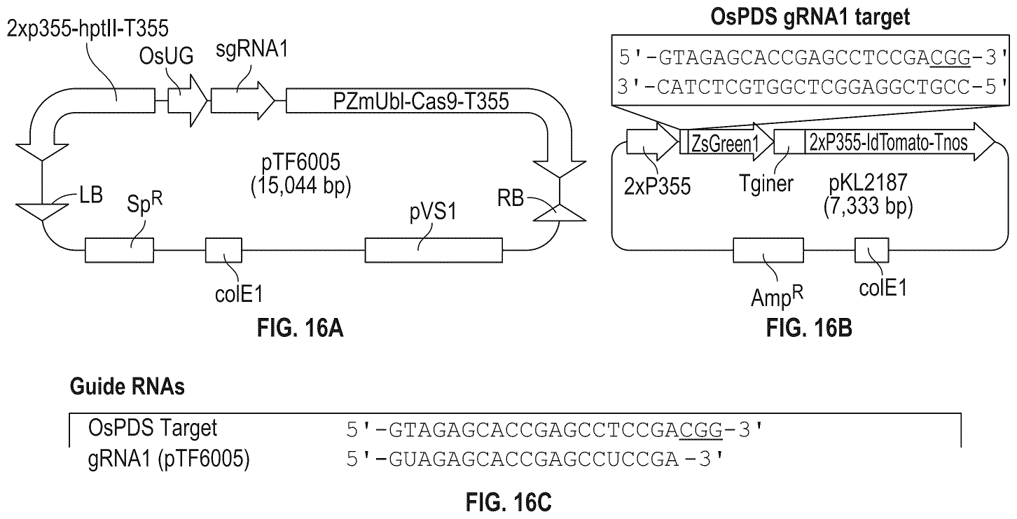

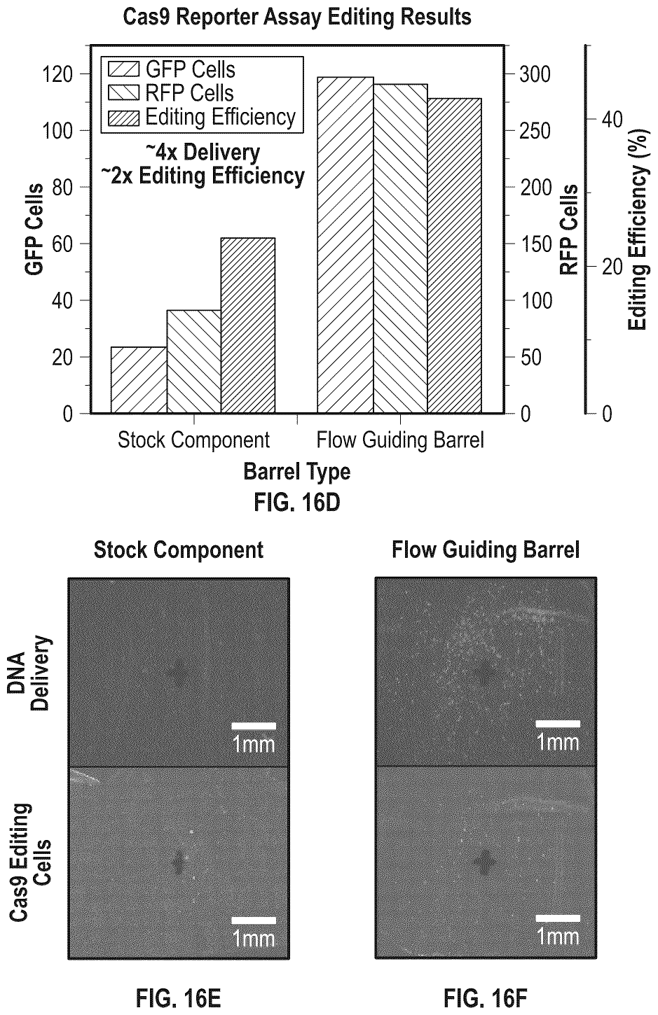

As shown in FIGS. 16A-F, on average, using the flow guiding barrel leads to at least 3-4 times (3-4×) more cells delivered to per shot (82 cells compared to 293 cells on average) as well as 2 times (2×) the editing efficiency (23.9% compared to 42.9% on average). The critical parameters of DNA and particle amount were kept constant for each bombardment, and plant tissue was sourced from the same onions. The distance between onion tissue and initial particle position was optimized for both conditions to account for the acceleration generated by increased confinement. The improvement in performance using Cas9 DNA due to the flow guiding barrel represents a significant upgrade that has broadly applicable potential.

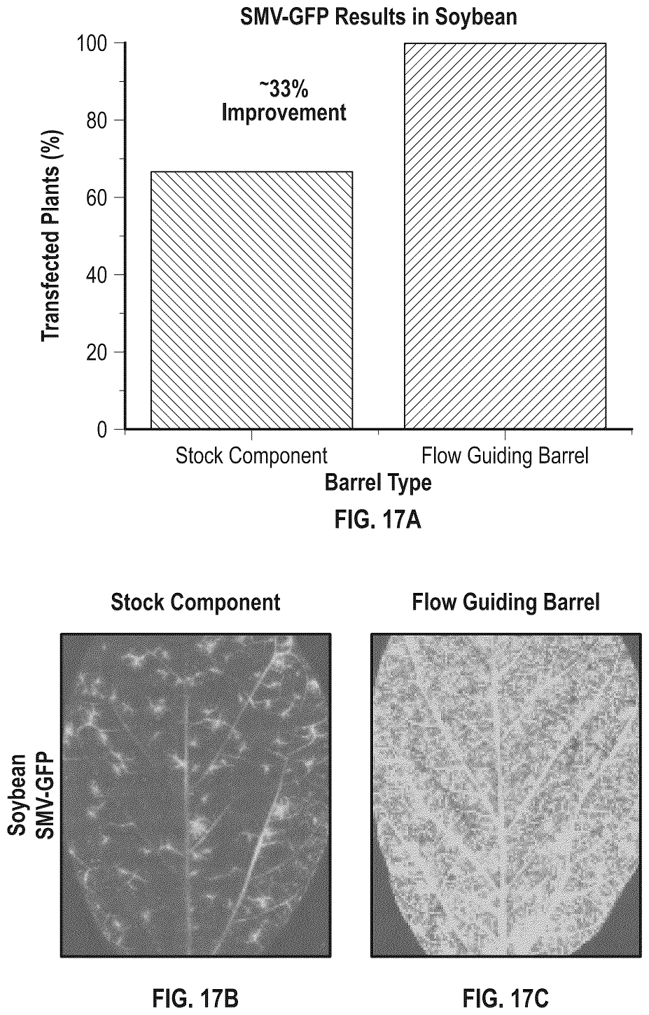

As shown in FIGS. 17A-C, on average, using the flow guiding barrel leads to 100% of the soybean plants being virally infected compared to 66% which is a significant improvement. The critical parameters of DNA and particle amount were kept constant for each bombardment, and plant tissue was sourced from the same onions. The distance between soybean tissue and initial particle position was optimized for both conditions to account for the acceleration generated by increased confinement. The improvement in performance using the soybean mosaic virus due to the flow guiding barrel represents a significant upgrade that can be applied to other plant species.

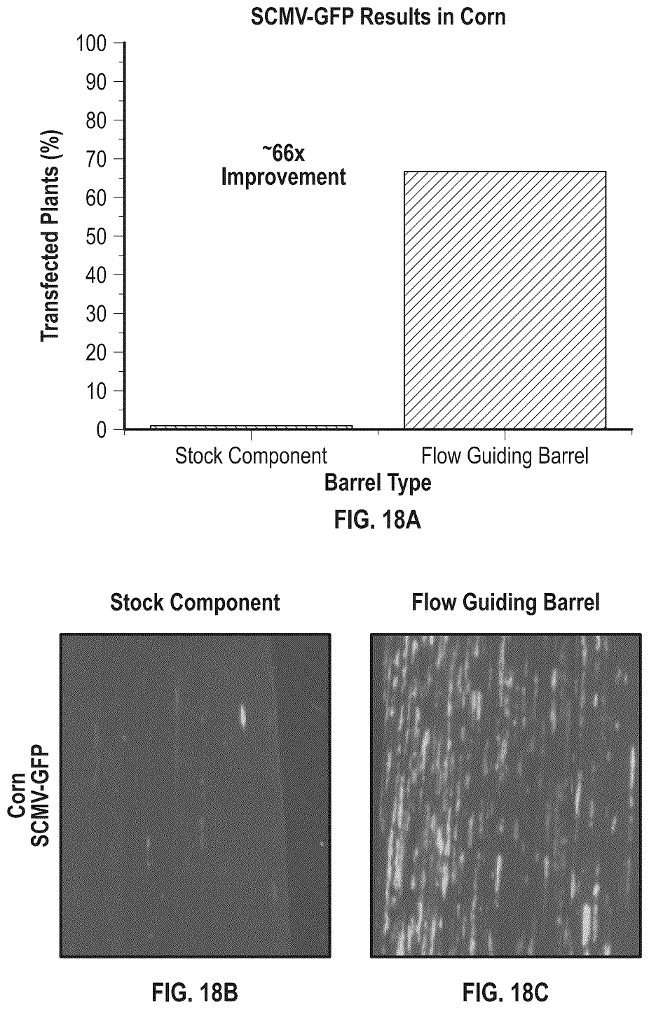

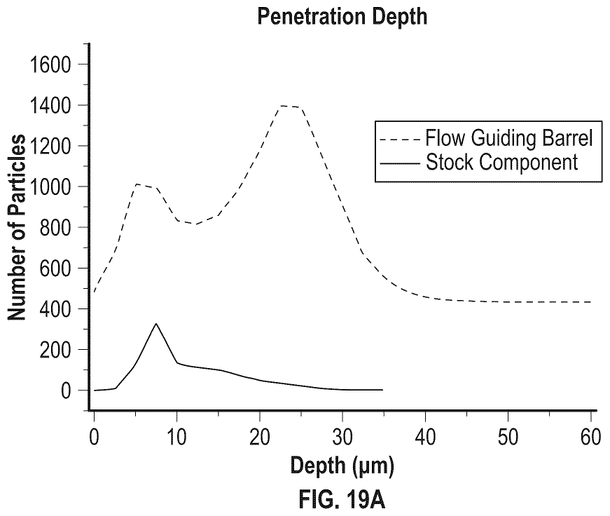

As shown in FIGS. 18A-C, on average, using the flow guiding barrel leads to at least 66 times (66×) as many plants being infected per experiment (66% compared to 1% infection efficiency). The critical parameters of DNA and particle amount were kept constant for each bombardment, and plant tissue was sourced from the same onions. The distance between corn tissue and initial particle position was optimized for both conditions to account for the acceleration generated by increased confinement. The improvement in performance using the sugarcane mosaic virus due to the flow guiding barrel represents a significant upgrade that can be applied to other plant species.

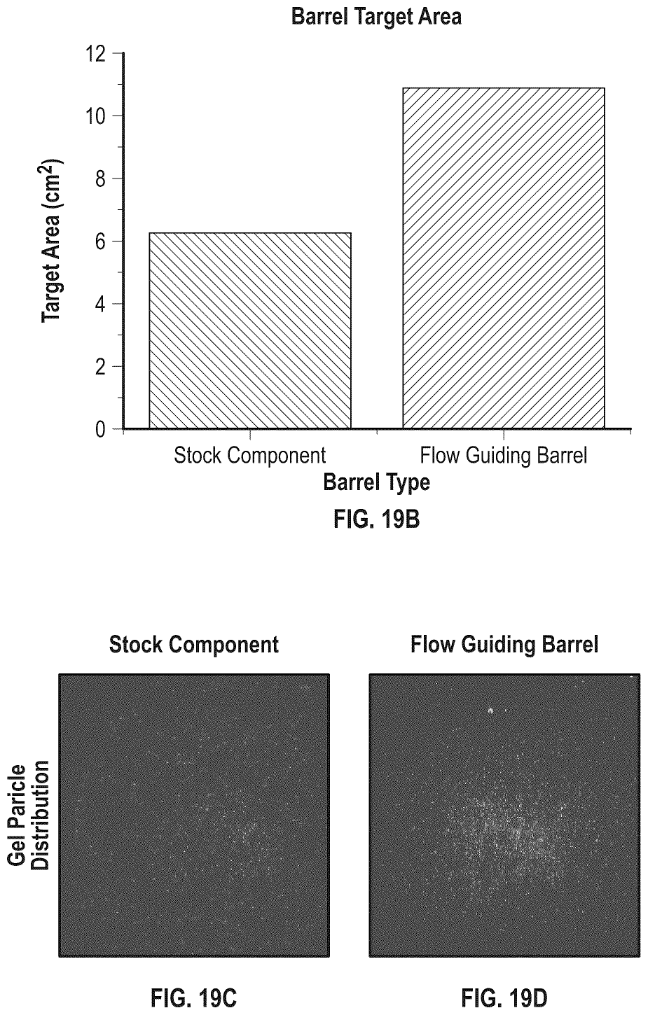

As shown in FIGS. 19A-D, on average, using the flow guiding barrel leads to 3 times (3×) the penetration depth (22.5 micrometers compared to 7.5 micrometers) and 2 times (2×) the particle distribution area (10.89 cm² compared to 6.25 cm²). The critical parameters of pressure, distance, and particle amount were kept constant for each bombardment. The distance between the agarose gel and initial particle position was optimized for both conditions to account for the acceleration generated by increased confinement.

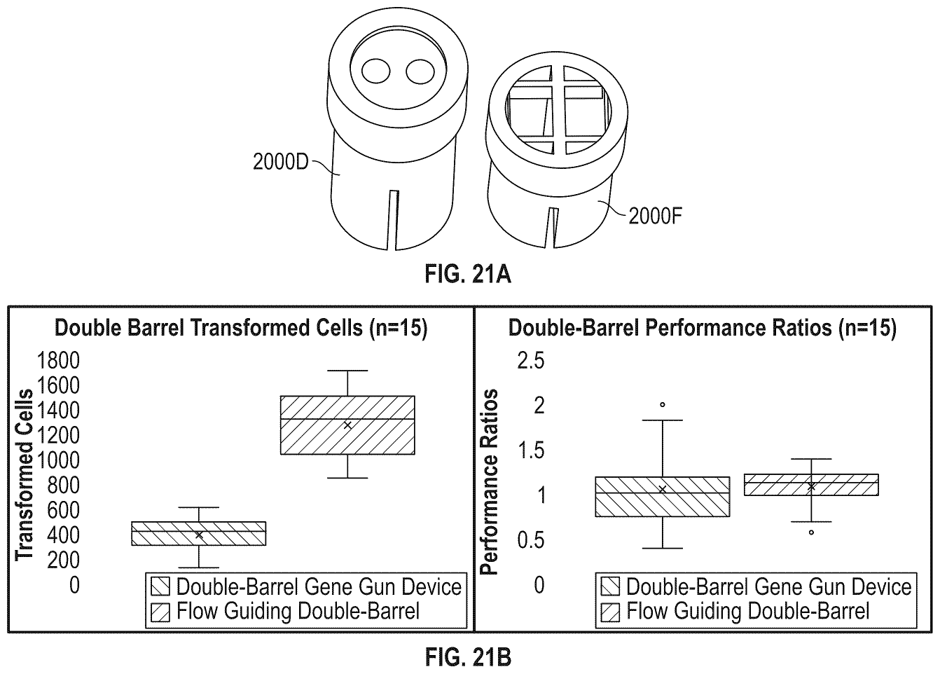

Barrels 2000D and 2000F were tested to compare use of a known barrel versus use of a flow-guiding double-barrel with respect to a total number of transformed cells and performance ratios. As shown in FIG. 21A, a first chart shows an improvement in number of cells transfected using the new design. As shown in FIG. 21B, a second chart measures the ability of the double-barrel to evaluate a consistent performance ratio, i.e. the number of cells transfected via the right barrel compared to the left. A performance ratio of 1 presents the real results, as both sides should be identical. The 3D images show the different barrel designs.

According to some examples, an optimal diameter of the barrel preferably ranges from thirteen to twenty-four millimeters (13 to 24 mm); more preferably ranges between eighteen and twenty-two millimeters (18 to 22 mm), and most preferably is approximately twenty millimeters (˜20 mm) in diameter.

According to some examples, an optimal length of the barrel preferably ranges from fifteen and eighty-five millimeters (15 to 85 mm), more preferably ranges between thirty and fifty-five millimeters (30 to 55 mm), and most preferably is approximately thirty-five millimeters (˜35 mm).

According to some examples, an optimal length of the barrel is preferably between one and five times (1× to 5×) greater than the diameter of the barrel, more preferably between one and a quarter times to three times (1.25× to 3×) greater than the diameter of the barrel, and most preferably between one and a half times and two times (1.5× to 2×) greater than the diameter of the barrel.

For our device that works universally for all applications, the ideal dimensions are a diameter of 20 mm and a length of 35 mm.

### LIST OF REFERENCE CHARACTERS

The following table of reference characters and descriptors are not exhaustive, nor limiting, and include reasonable equivalents. If possible, elements identified by a reference character below and/or those elements which are near ubiquitous within the art can replace or supplement any element identified by another reference character.

TABLE 1
List of Reference Characters

| | |
|---|---|
| 100 | system |
| 102 | bombardment chamber |
| 104 | bombardment chamber door |
| 106 | rupture disk retaining cap |
| 108 | launch assembly shelf |
| 110 | vacuum tubing |
| 112 | tank |
| 114 | helium regulator |
| 116 | helium pressure regulator |
| 118 | a solenoid valve |
| 120 | connective polyether ether ketone (“PEEK”) tubing |
| 122 | on/off power switch |
| 124 | fire switch |
| 126 | vacuum/vent/hold switch assembly |
| 128 | vacuum gauge |
| 130 | vacuum/vent rate control valves |
| 132 | helium pressure gauge |
| 200 | microcarrier launch assembly |
| 200S | stock component |
| 202 | macrocarrier cover lid |
| 204 | an adjustable nest |
| 206 | a fixed nest |
| 208 | a stopping screen support ring |
| 210 | two spacer rings |
| 212 | macrocarrier holders |
| 300 | plate shelf |
| 400 | ballistic process |
| 402 | high-pressure helium |
| 404 | a rupture disk |
| 406 | gas acceleration tube |
| 408 | macrocarrier sheet |
| 410 | microscopic gold microcarriers |
| 412 | target cells |
| 414 | stopping screen |
| 416 | distance from the rupture disk to the macrocarrier sheet |
| 418 | macrocarrier travel distance to the stopping screen |
| 420 | distance between the stopping screen and the target cells |
| 500 | portable, helium driven gene gun |
| 502 | cartridge |
| 504 | cylinder lock |
| 506 | barrel pin |
| 508 | cartridge holder |
| 510 | O-ring |
| 512 | cylinder advance lever |
| 514 | barrel liner |
| 516 | safety interlock switch |
| 518 | trigger button |
| 520 | LED display |
| 522 | metal bar |
| 524 | battery access cover |
| 526 | battery compartment |
| 528 | terminals |
| 530 | attachment fitting (male) |
| 532 | acceleration chamber |
| 534 | spacer |
| 600 | helium regulator |
| 602 | connection to helium tank |
| 604 | sleeve |
| 606 | female quick connect fitting |
| 608 | pressure relief valve |
| 610 | high velocity stream of helium |
| 700 | helium hose |
| 702 | male quick connect fitting |
| 704 | female quick connect fitting |
| 100P | position at which stock component can be replaced |
| | for flow guiding barrels 800, 900, 1000 |
| 800 | single-flow guiding barrel |
| 802 | body |
| 804 | retaining ring |
| 806 | major flow path |
| 808 | peripheral, minor flow paths |
| 900 | double-flow guiding barrel |
| 902 | body |
| 904 | retaining ring |
| 906A | first major flow path |
| 906B | second major flow path |
| 908 | peripheral, minor flow paths |
| 910 | major divider |
| 1000 | multi-flow guiding barrel |
| 1002 | body |
| 1004 | retaining ring |
| 1006A | first major flow path |
| 1006B | second major flow path |
| 1006C | third major flow path |
| 1006D | fourth major flow path |
| 1010 | major dividers |
| 1100A | shorter barrel |
| 1100B | longer barrel |
| 1100C | smaller flow path |
| 1100D | larger flow path |
| 2000A | stock component |
| 2000B | short, flow-guiding single barrel |
| 2000C | long, flow-guiding single barrel |
| 2000D | narrow double barrel |
| 2000E | wide double barrel |
| 2000F | double barrel with a single fin |
| 2000G | multi-barrel |
| 2000H | short, flow-guiding multi-barrel |
| 2000I | long, flow-guiding multi-barrel |
| 2000J | flow-guiding multi-flow barrel |
| 2000K | flow-guiding multi-flow barrel with a single fin |
| 2000L | flow-guiding multi-flow barrel with a plurality of fins |

### Glossary

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

As used herein, the term “exemplary” refers to an example, an instance, or an illustration, and does not indicate a most preferred embodiment unless otherwise stated.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. An attachment for a biolistic particle delivery system that confines the flow of particles to a barrel comprising:
an elongated body with a central cavity that defines a major flow path passing through; and
a ring located at a top of the cylindrical body, said ring having a diameter greater than a diameter of the cylindrical body;
wherein the ring is retrofit to a macrocarrier launch assembly of the biolistic particle delivery system.

2. The attachment of claim 1 wherein the elongated body is a hollow cylinder with a length that is between one and a half times and two times (1.5× to 2×) greater than a diameter of the elongated body.

3. The attachment of claim 1 wherein the elongated body tapers from an initial thickness greatest at the location where the ring meets the top of the cylindrical body to a terminal thickness less than the initial thickness.

4. The attachment of claim 1 wherein the major flow path is divided into more than one major flow path by one or more dividers.

5. The attachment of claim 4 wherein the one or more dividers begin at the top of the cylindrical body and extend only to a depth at or within the elongated body.

6. The attachment of claim 5 wherein each of the more than one major flow path is defined by a cavity shape having at least one curved wall and at least one planar wall.

7. The attachment of claim 4 wherein the one or more dividers begin at the top of the cylindrical body and extend to a depth beyond the elongated body, thereby acting as fins to help guide flow even as particles have exited the central cavity.

8. The attachment of claim 1 further comprising minor flow paths located adjacent a periphery of the central cavity, said major flow path separated from the minor flow paths by one or more baffles.

9. The attachment of claim 1 wherein flow of the major flow path is substantially laminar.

10. The attachment of claim 1 wherein the attachment is symmetrical about at least an x-axis and a y-axis and is asymmetrical about a z-axis, said z-axis being defined by an axial axis traversing a longitude of the elongated body.

11. A biolistic particle delivery system comprising:
a gas acceleration tube;
a source of high-pressure gas operatively connected to a first end of the gas acceleration tube; and
the attachment of claim 1 located at a second end of the gas acceleration tube.

12. The biolistic particle delivery system of claim 11 further comprising a screen that allows only the biolistic particles to impact an intended target.

13. The biolistic particle delivery system of claim 11 wherein the biolistic particle delivery system comprises a bombardment chamber, connective tubing attached to a vacuum source, a helium regulator, and a solenoid valve.

14. The biolistic particle delivery system of claim 13 further comprising a rupture disk.

15. The biolistic particle delivery system of claim 11 wherein the biolistic particle delivery system is a helium driven gene gun.

16. A method of delivering biolistic particles comprising:
using a high-pressure gas to move biolistic particles through an attachment for a biolistic particle delivery system toward a target having one or more cells selected from the group consisting of: bacterial cells, fungal cells, insect cells, plant cells, animal cells, intracellular organelles, and combinations thereof; wherein the attachment comprises an elongated barrel; and
bombarding the targets with coated microcarriers.

17. The method of claim 16 further comprising replacing a stock component with the elongated barrel.

18. The method of claim 17 further comprising reducing a number of outlier particles through use of the stock component by employing said elongated barrel in lieu thereof.

19. The method of claim 17 further comprising reducing a number of cells killed through use of the stock component by employing said elongated barrel in lieu thereof.

20. The method of claim 17 further comprising increasing an efficacy related to penetration of a cell wall.


SEQUENCE LISTING (3 sequences)

SEQ ID NO: 1 - DNA, 23 bp, synthetic construct
  source 1..23: mol_type = other DNA
gtagagcaccgagcctccgacgg

SEQ ID NO: 2 - DNA, 23 bp, synthetic construct
  source 1..23: mol_type = other DNA
catctcgtggctcggaggctgcc

SEQ ID NO: 3 - RNA, 20 nt, synthetic construct
  source 1..20: mol_type = other RNA
gtagagcaccgagcctccga